(19) United States
(12) Patent Application Publication
(10) Pub. No.: US 2019/0023764 A1
(43) Pub. Date: Jan. 24, 2019

(54) CAR HAVING REPLICATED BINDING MOTIFS IN A CO-STIMULATORY DOMAIN

(71) Applicants: ProMab Biotechnologies, Inc., Richmond, CA (US); Forevertek Biotechnology Co., Ltd., Changsha (CN)

(72) Inventors: Lijun WU, Albany, CA (US); Vita GOLUBOVSKAYA, Richmond, CA (US); Hua ZHOU, Hercules, CA (US)

(21) Appl. No.: 16/151,233

(22) Filed: Oct. 3, 2018

Related U.S. Application Data
(63) Continuation of application No. PCT/US2017/024755, filed on Mar. 29, 2017.
(60) Provisional application No. 62/317,975, filed on Apr. 4, 2016.

(51) Int. Cl. C07K 14/705; C12N 15/62

(52) U.S. Cl. CPC C07K 14/70578; C12N 5/0638; C07K 14/70521; C12N 15/62

(57) ABSTRACT

The present invention is directed to a chimeric antigen receptor fusion protein comprising from N-terminus to C-terminus: (i) a single-chain variable fragment (scFv) having activity against tumor antigen, (ii) a transmembrane domain, (iii) at least one co-stimulatory domain having one or more binding motifs immediately repeated at least one time, and (iv) an activating domain. A preferred co-stimulatory domain is derived from human CD28, 4-1BB, ICOS-1, CD27, OX-40, GITR, or DAP10.

This application is a continuation of PCT/US2017/024755, filed Mar. 29, 2017; which claims the priority of U.S. Provisional Application No. 62/317,975, filed Apr. 4, 2016. The contents of the above-identified applications are incorporated herein by reference in their entireties.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Mar. 24, 2017, and a size of 39.5 kilobytes. The Sequence Listing filed via EFS-Web is part of the specification and is hereby incorporated in its entirety by reference herein.

### FIELD OF THE INVENTION

The present invention relates to a chimeric antigen receptor comprising mutations in the co-stimulating domain. The invention particularly relates to chimeric antigen receptor-T cells having replicated activation motifs in the co-stimulatory domains.

### BACKGROUND OF THE INVENTION

Immunotherapy is emerging as a highly promising approach for the treatment of cancer. T cells or T lymphocytes are the armed forces of our immune system that constantly look for foreign antigens and discriminates abnormal (cancer or infected cells) from normal cells. Genetically modifying T cells with CARs are a common approach to design tumor-specific T cells. CAR-T cells targeting tumor-associated antigens can be infused into patients (called adoptive cell transfer or ACT) representing an efficient immunotherapy approach. The advantage of CAR-T technology compared with chemotherapy or antibody is that reprogrammed engineered T cells can proliferate and persist in the patient and work like a living drug.

CARs (Chimeric antigen receptors) usually consist of a monoclonal antibody-derived single-chain variable fragment (scFv) linked by a hinge and transmembrane domain to a variable number of intracellular signaling domains and a single, cellular activating, CD3-zeta domain.

FIG. 1 shows the evolution of CARs from first generation (left, with no co-stimulation domains) to second generation (middle, with one co-stimulation domain CD28 or 4-BB) to third generation (with two or several co-stimulation domains), see Golubovskaya, Wu, Cancers, 2016 Mar. 15; 8(3). Generating CARs with multiple costimulatory domains (third generation CAR) have led to increased cytolytic activity, and significantly improved persistence of CAR-T cells that demonstrate augmented antitumor activity.

### BRIEF DESCRIPTION OF THE DRAWINGS

FIG. 1 shows the structure of CAR from first to third generation.

FIG. 2 shows the structures of Mesothelin CAR and EGFR CAR with no mutation and with mutations of CD28 co-activation domain.

FIG. 3 shows that mesothelin CAR-T cells with mutant CD28 domain are highly cytolytic against mesothelin-positive ovarian cancer cells.

FIG. 4 shows that mesothelin CAR-T cells with mutant CD28 domain are not cytolytic against mesothelin-negative cancer cells (C30 cells).

FIG. 5 shows that mesothelin CAR-T cells with mutant CD28 domain are highly cytolytic against mesothelin-positive pancreatic cancer cells.

FIG. 6 shows that EGFR CAR-T cells with mutant CD28 domain are highly cytolytic against EGFR-positive ovarian cancer cells.

FIG. 7 shows secretion of IFN-gamma by target cells alone, T cells, Mock CAR-T, Meso-TM28-28 WT-Z CAR-T, Meso-TM28-28 DD-Z CAR-T, and Meso-Flag-TM28-28 DD-Z CAR-T against A1847 cells.

FIG. 8 shows secretion of IL-2 by target cells alone, T cells, Mock CAR-T, Meso-TM28-28 WT-Z CAR-T, Meso-TM28-28 DD-Z CAR-T, and Meso-Flag-TM28-28 DD-Z CAR-T against A1847 cells.

FIG. 9 shows secretion of IL-6 by target cells alone, T cells, Mock CAR-T, Meso-TM28-28 WT-Z CAR-T, Meso-TM28-28 DD-Z CAR-T, and Meso-Flag-TM28-28 DD-Z CAR-T against A1847 cells.

FIG. 10 shows secretion of IFN-gamma by T cells, Mock CAR-T, EGFR-TM28-28 WT-Z, EGFR-TM28-28 DDT195P-Z cells against BxPC3 cells.

FIG. 11 shows secretion of IL-2 by T cells, Mock CAR-T, EGFR-TM28-28 WT-Z, EGFR-TM28-28 DDT195P-Z cells against BxPC3 cells.

FIG. 12 shows secretion of IL-6 by T cells, Mock CAR-T, EGFR-TM28-28 WT-Z, EGFR-TM28-28 DDT195P-Z cells against BxPC3 cells.

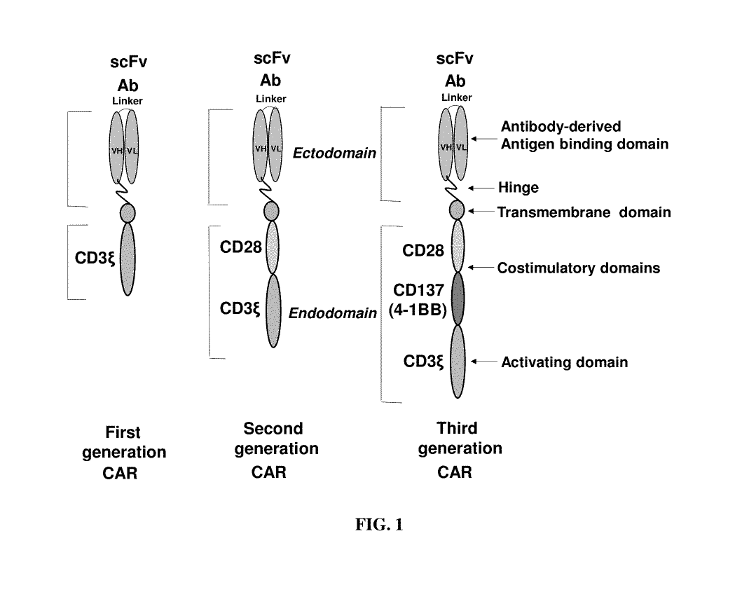

### DETAILED DESCRIPTION OF THE INVENTION

### Definitions

As used herein, a “chimeric antigen receptor (CAR)” means a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, a FLAG-tag, or FLAG octapeptide, or FLAG epitope, is a polypeptide protein tag that can be added to a protein using recombinant DNA technology, having the sequence motif DYKDDDDK (SEQ ID NO; 1). It can be fused to the C-terminus or the N-terminus of a protein, or inserted within a protein.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for preparing an scFv are known to a person skilled in the art.

As used herein, a “tumor antigen” means a biological molecule having antigenecity, expression of which causes cancer.

### DESCRIPTION

The inventors have discovered that by introducing one or more specific mutations in the binding motifs of a co-stimulating domain of a chimeric antigen receptor (CAR), the cytotoxicity of CAR-T cells against target cells can be increased. Thus the specific mutations increase functional activities of CAR-T cells, which attack tumor cells more effectively. The inventors have also discovered that CAR-T cells having such mutated CAR in general produce less interferon (IFN)-γ, and may produce less interleukin-2 (IL-2) and less interleukin-6 (IL-6), than the corresponding wild-type CAR-T cells, which indicates that the mutated CAR-T cells can be less toxic and safer than the corresponding wild-type CAR-T cells.

The present invention is directed to a chimeric antigen receptor fusion protein comprising from N-terminus to C-terminus: (i) a single-chain variable fragment (scFv) having activity against a tumor antigen, (ii) a transmembrane domain, (iii) at least one co-stimulatory domain having one or more binding motif immediately repeated at least one time, and (iv) an activating domain.

In one embodiment, the tumor antigen is selected from the group consisting of: mesothelin, BCMA, VEGFR-2, CD4, CD5, CD19, CD20, CD30, CD22, CD24, CD25, CD28, CD30, CD33, CD38, CD47, CD52, CD56, CD80, CD81, CD86, CD123, CD138, CD171, CD276, B7H4, CD133, EGFR, GPC3; PMSA, CD3, CEACAM6, c-Met, EGFRvIII, ErbB2/HER-2, ErbB3/HER3, ErbB4/HER-4, EphA2,10a, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, NGFR, MCAM, TNFR1, TNFR2, PD1, PD-L1, PD-L2, HVEM, MAGE-A, NY-ESO-1, PSMA, RANK, ROR1, ROR-2, TNFRSF4, CD40, CD137, TWEAK-R, LTPR, LIFRP, LRP5, MUC1, TCRa, TCRp, TLR7, TLR9, PTCH1, WT-1, Robol, a, Frizzled, OX40, CD79b, and Notch-1-4.

In one embodiment, the tumor antigen is mesothelin. Mesothelin is a 40 kDa protein present on normal mesothelial cells and overexpressed in several human tumors, including mesothelioma and ovarian and pancreatic adenocarcinoma.

In one embodiment, the tumor antigen is epidermal growth factor receptor (EGFR, ErbB-1, HER1 in humans). EGFR is a transmembrane protein that is a receptor for members of the epidermal growth factor family (EGF family) of extracellular protein ligands.

The CAR of the present invention comprises a single chain variable fragment (scFv) that binds specifically to the tumor antigen of interest. The heavy chain (H chain) and light chain (L chain) fragments of an antibody are linked via a linker sequence. For example, a linker can be 5-20 amino acids. For example, a linker sequence having a glycine-serine (GGGGS) repeated several times as a continuous sequence can be used. The scFv structure can be VL-linker-VH, or VH-linker-VL, from N-terminus to C-terminus.

The CAR of the present invention comprises a transmembrane domain which spans the membrane. The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. In preferred embodiments, the transmembrane domain is derived from CD28 or CD8, which give good receptor stability.

In the present invention, the co-stimulatory domain that has one or more mutations is selected from the group consisting of human CD28, 4-1BB (CD137), ICOS-1, CD27, OX 40 (CD137), DAP10, and GITR (AITR). The cytoplasmic domain, protein binding sites and activation sites (binding motifs) of the above co-stimulatory proteins are shown in Table 1 below.

TABLE 1

| Costimulatory domain | Cytoplasmic domain, protein | |
|---|---|---|
| Length aa, MW kDa | binding and activation sites | Reference |
| CD28 | 191-194 YMNM, site of | Boomer et al. Cold |
| 220 aa, 25 kDa | interaction with PI3K, Grb2 | Spring Harb |
| | 208-211 PYAP, site of interaction | Perspect Biol 2010, |
| | with Lck, Grb2 | 2, a002436 |
| | T195P (Threonine 195)-activating | |
| | mutation | |
| | (SEQ ID NO: 2 and 3) | |
| 4-1BB (CD137) | 214-255 aa, cytoplasmic domain | Robert H. Arch et al. |
| 255 aa, 27.8 kDa | Interacts with TRAF1, TRAF2 and | Molecular and |
| | TRAF3. Interacts with LRR-repeat | Cellular Biology, |
| | protein 1/LRR-1. | V18, 1, 1998, p. |
| | LRR-1, 214-255 interaction | 558-565 |
| | 236-239, QEED and | |
| | 247-250, EEEE, sites of | |
| | interaction with TRAF | |
| | proteins1, 2, 3 increasing | |
| | intracellular signaling (NF-kappa B | |
| | signaling) | |
| | (SEQ ID NOs: 4 and 5) | |
| ICOS (CD278) | 162-199 aa, cytoplasmic domain 7 | Rudd, Schneider H, |
| 199 aa, 22.6 kDa | interactions: Ligands: B7-H2, | Nature Reviews |
| | B7H2, B7RP-1, B7RP1, CD275, | Immunology 3, 544-556 |
| | GL50, ICOS-L, ICOSL, LICOS. | (July 2003) |
| | Interacts with TNF, ICOS, Pi3K | |
| | 180 YMFM 183, PI3K Kinase | |
| | binding | |
| | (SEQ ID NO: 6) | |
| OX40 (CD134) | 236-277 aa, cytoplasmic domain- | Robert H. Arch et al. |
| 277 aa, 29.3 kDa | Interacts with TRAF2, TRAF3 and | Molecular and |
| | TRAF5 | Cellular Biology, |
| | 261-269 aa, 261 TPIQEEQAD | V18, 1, 1998, p. |
| | 269, site of interaction with TRAF | 558-565 |
| | proteins, activating NF-kappa B | |
| | signaling | |
| | (SEQ ID NO: 7) | |
| DAP10 (KAP10) | 70-93 aa, cytoplasmic domain, | Upshaw J L et al, |
| 93 aa, 9.4 kDa | Interacts with PIK3R1 and GRB2, | Nature Immunology |
| | KLRK1 (through transmembrane | 7, 524-532 (2006) |
| | domain, induces NK cytotoxicity), | |
| | ELAV-1 | |
| | 86 YINM 89-binds PIK3R | |
| | 88, N → Q: Abrogates cell killing | |
| | and interaction with GRB2. No | |
| | effect on interaction with PIK3R1 | |
| | 89, M → Q: Abrogates cell killing | |
| | and interaction with PIK3R1. No | |
| | effect on interaction with GRB2 | |
| | (SEQ ID NO: 8) | |
| CD27 | 213-260 aa, cytoplasmic domain, | Hisaya Akiba et al, |
| 260 aa, 29.1 kDa | iteracts with TRAF TRAF 1, TRAF | JBC, 273, 13353-13358, |
| | 2, TRAF 3, TRAF5, SIVA1, | 1998 |
| | CD70, HRAS, BIRC2. | |
| | 246 PIQED 250, TRAF binding | |
| | site | |
| | (SEQ ID NO: 9) | |
| GITR(AITR) | 184-241 aa, cytoplasmic domain | Ludovic Tibor |
| Tumor necrosis factor | 217 SCQFPEEERGE 227, TRAF | Krausz, et al, The |
| receptor superfamily | binding site | Scientific World |
| member 18 | Binds to TRAF1, TRAF2, and | JOURNAL, (2007) |
| 255 aa, 26.8 kDa | TRAF3, but not TRAF5 and | 7, 533-566 |
| | TRAF6. Binds through its C- | |
| | terminus to SIVA1/SIVA | |
| | (SEQ ID NO: 10) | |

In one embodiment, the co-stimulatory domain is CD28. The fusion protein comprises the binding motif YMNM (191-194) immediately repeated 1, 2, 3, or 4 times; preferably one time. In another embodiment, the fusion protein comprises the binding motif PYAP immediately repeated 1, 2, 3, or 4 times; preferably one time. “Immediately repeated” refers that there is no other amino acid in between YMNM and YMNM (SEQ ID NO: 2), or in between PYAP and PYAP (SEQ ID NO: 3). In yet another embodiment, the fusion protein further comprises a mutation from T to P immediately after the last repeat of YMNM. The T to P mutation (T195P) corresponds to amino acid position 195 of the CD28 protein. In yet another embodiment, the fusion protein comprises double mutations, i.e., the binding motifs YMNM and PYAP are both immediately repeated once, optionally further comprises mutation from T to P immediately after the last repeat of YMNM.

In another embodiment, the co-stimulatory domain is 4-1BB (CD137), ICOS-1, CD27, OX 40 (CD137), DAP10, or GITR (AITR), and its binding motif can be immediately repeated 1, 2, 3, or 4 times, as described above for CD28, to increase its activity. Further, a mutation site can be created in these co-stimulatory domains to increase the activity.

The endodomain (the activating domain) is the signal-transmission portion of the CAR. After antigen recognition, receptors cluster and a signal is transmitted to the cell. The most commonly used endodomain component is that of CD3-zeta (CD3 Z or CD3), which contains 3 ITAMs. This transmits an activation signal to the T cell after antigen is bound. CD3-zeta may not provide a fully competent activation signal and additional co-stimulatory signaling may be needed. For example, one or more co-stimulating domains listed in Table 1 can be used with CD3-Zeta to transmit a proliferative/survival signal.

In one embodiment of the invention, the fusion protein further comprises a FLAG tag N-terminus to V_{H}, or between V_{H} and V_{L}, or between V_{L} and the transmembrane domain. The FLAG tag needs to be in extracellular domain, and not in the intracellular domain. In addition to FLAG tag, other tags can be used in the construct. Strep tag, His tag, Xpress tag, Avi tag, Calmodulin tag, Polyglutamate tag, HA tag, Myc tag, Nus tag, S tag, X tag, SBP tag, Softag, V5 tag, CBP, GST, MBP, GFP, Thioredoxin tag, or any combination can be used in many biological applications (toxin-conjugated antibodies, in vivo cell imaging, tag-magnetic beads cell sorting for cell expansion, etc).

The CAR of the present invention may comprise a signal peptide N-terminal to the ScFv so that when the CAR is expressed inside a cell, such as a T-cell, the nascent protein is directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. As an example, the signal peptide may derive from human CD8 or GM-CSF, or a variant thereof having 1 or 2 amino acid mutations provided that the signal peptide still functions to cause cell surface expression of the CAR.

The CAR of the present invention may comprise a spacer sequence as a hinge to connect scFv with the transmembrane domain and spatially separate antigen binding domain from the endodomain. A flexible spacer allows to the binding domain to orient in different directions to enable its binding to a tumor antigen. The spacer sequence may, for example, comprise an IgG1 Fc region, an IgG1 hinge or a CD8 stalk, or a combination thereof. A human CD8 stalk is preferred.

In the present invention, the genetic modification of CD28 co-activation CAR domain is performed by duplicating activation CD28 regions and an optional insertion of the point mutation T195P, which may increase binding of CD28 partners: GRB2 and GADS/GRAP2. CD 28 protein has two motifs: YMNM and PYAP, responsible for binding of its partners and down-stream activity. First in natural form of CD28, CD80 and CD86 on antigen-presenting cell (APC) binds CD28 extracellular domain, then this interaction initiates signal transduction cascade starts inside cytoplasmic tail of CD28. In the present invention, mesothelin binds extracellular Mesothelin ScFv and transmits the signaling to CD28 intracellular co-activation domain. Src kinase phosphorylates tyrosine in YMNM motif that cause binding of Grb2 and PI3K activating down-stream signaling. A second binding motif PYAP of CD28 binds different set of partners: Lck, Grb-2 and Filamin A. PYAP motif may be critical for CD28-dependent IL-2 production.

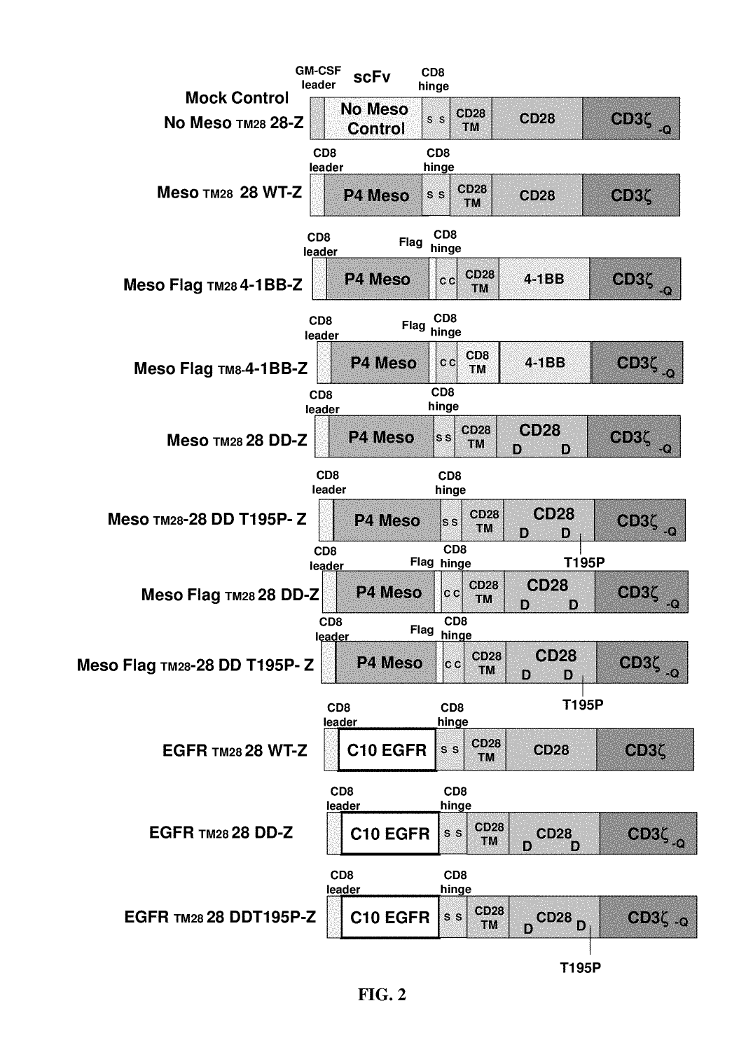

FIG. 2 shows the structures of Mesothelin CAR and EGFR CAR with no mutation and with mutations of CD28 co-activation domain. The Mesothelin scFv or EGFR scFv C10 antibodies are used. The CD28 without mutation is marked as 28 WT, with mutations of duplicated motif YMNM (191-194 amino acids) and also duplicated motif PYAP (208-211 amino-acids), is marked as 28 DD (double duplicated motif). Some DD mutations have an additional point mutation T195P, which is marked as 28 DDT195P. The same mutant CD28 domain is used with EGFR scFV. The Mesothelin constructs are either without Flag or with Flag tag after Mesothelin ScFv. The Mesothelin Flag CAR constructs contained two serines changed to cysteines in the CD8 hinge region (marked C C). CD3 zeta domain is marked as Z. No Meso control targets intracellular protein and is used as mock no mesothelin control; -Q shows absence of Gln in the CD3 zeta domain. GM-CSF leader sequence was used for Mock control constructs, and human CD 8 leader sequence was used for Meso and EGFR CAR constructs.

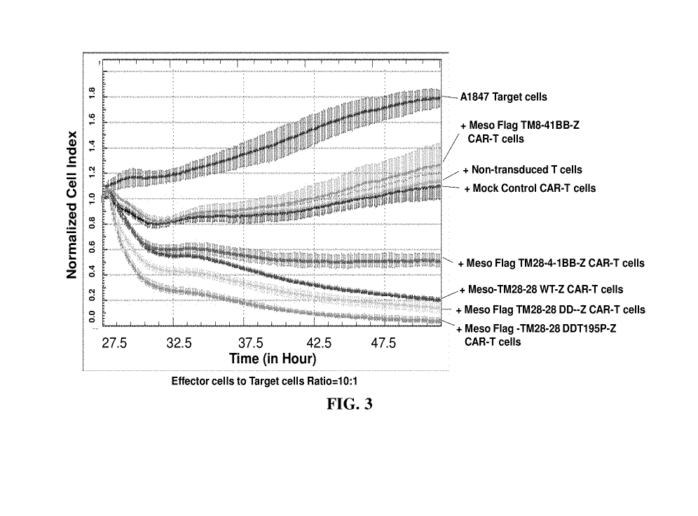

Since both CD28 motifs (YMNM and PYAP) are critical for its down-stream signaling, the inventors have duplicated each motif of CD28 responsible for binding of its partners and IL-2 production and introduced inside anti-mesothelin-CAR construct. Another invention is to introduce T195P mutation responsible for binding of Grb2 partner and activation of CD28. The inventors have generated one CD28 mutant with duplicated motifs YMNM-YMNM and PYAP-PYAP motif (FIG. 3, 5th panel), and have also generated CD28 mutant with double motifs plus T195P point mutation immediately after the second YMNM (FIG. 3, last panel). The inventors compared activity of these CAR constructs with wild type CD28 CAR and wild type 4-1BB-Z CAR targeting mesothelin constructs (FIG. 3). The inventors have introduced FLAG tag after mesothelin scFv. The inventors have also prepared same mutant constructs without FLAG-tag at the C-terminal domain of mesothelin scFv.

The present invention provides a nucleic acid encoding the CAR described above. The nucleic acid encoding the CAR can be easily prepared from an amino acid sequence of the specified CAR by a conventional method. A base sequence encoding an amino acid sequence can be obtained from the aforementioned NCBI RefSeq IDs or accession numbers of GenBenk for an amino acid sequence of each domain, and the nucleic acid of the present invention can be prepared using a standard molecular biological and/or chemical procedure. For example, based on the base sequence, a nucleic acid can be synthesized, and the nucleic acid of the present invention can be prepared by combining DNA fragments which are obtained from a cDNA library using a polymerase chain reaction (PCR).

The nucleic acid encoding the CAR of the present invention can be inserted into a vector, and the vector can be introduced into a cell. For example, a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a Sendai virus vector, an Epstein-Barr virus (EBV) vector, and a HSV vector can be used. As the virus vector, a virus vector lacking the replicating ability so as not to self-replicate in an infected cell is preferably used.

For example, when a retrovirus vector is used, the process of the present invention can be carried out by selecting a suitable packaging cell based on a LTR sequence and a packaging signal sequence possessed by the vector and preparing a retrovirus particle using the packaging cell. Examples of the packaging cell include PG13 (ATCC CRL-10686), PA317 (ATCC CRL-9078), GP+E-86 and GP+envAm-12, and Psi-Crip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies.

The present invention provides T cells modified to express the chimeric antigen receptor fusion protein as described above. CAR-T cells of the present invention bind to a specific antigen via the CAR, thereby a signal is transmitted into the cell, and as a result, the cell is activated. The activation of the cell expressing the CAR is varied depending on the kind of a host cell and an intracellular domain of the CAR, and can be confirmed based on, for example, release of a cytokine, improvement of a cell proliferation rate, change in a cell surface molecule, or the like as an index. For example, release of certain cytotoxic cytokines (a tumor necrosis factor, lymphotoxin, etc.) from the activated cell causes destruction of a target cell expressing an antigen. In addition, release of a cytokine or change in a cell surface molecule stimulates other immune cells, for example, a B cell, a dendritic cell, a NK cell, and a macrophage.

T cells modified to express the CAR can be used as a therapeutic agent for a disease. The therapeutic agent comprises the cell expressing the CAR as an active ingredient, and may further comprise a suitable excipient. Examples of the excipient include pharmaceutically acceptable excipients known to a person skilled in the art.

The inventors have generated CAR comprising genetically modified CD28 co-activation domain with anti-mesothelin scFv and anti-EGFR scFv. The CARs comprise duplicated activation CD28 motifs, which are responsible for binding of co-activation partners of CD28 such as PI3K and other partners regulating IL-2 secretion upon activation of T cells. In another construct, the inventors have added additional single point mutation: T195P (of CD28 protein), which also can increase binding of CD28 partners GRB-2 and GADS/GRAP2 to its cytoplasmic tail.

The data demonstrate efficient growth of T cells transduced with these mutant constructs. The data also demonstrate that mesothelin CD28DD-Z CAR-T and mesothelin CD28DDT195P28-Z cells expanded effectively in vitro, and expressed better or similar cytolytic activity compared with mesothelin CD28 wild type-Z CAR-T cells against mesothelin-positive cancer cells. Real-time cytotoxicity assay with ovarian cancer cell line and pancreatic cancer cell line overexpressing mesothelin demonstrated highest activity of CD28 domain double-mutation (DD) plus T195P mutation, followed by double mutation, and then by wild type mesothelin. The data also show that CARs comprising CD28 mutant significantly decreased cytokine secretion in case of mesothelin-28DD-Z CAR-T cells, and differentially regulated in case of EGFR-28DDT195P-Z CAR-T cells.

One advantage of genetically modified and engineered CAR-T cells containing mutant CD28 co-activation domain of the present invention is their increased functional activities (e.g., cytotoxicity against target cells) of CAR-T cells compared with those of the wild type mesothelin. This method of CD28 modification, which optimizes CAR-T functions, has a high potential for use in pre-clinical and clinical trials. Another advantage of CAR-T cells containing mutant CD28 co-activation domain of the present invention is reduced cytokine secretion. Thus, the CAR-T cells of the present invention may be less toxic in terms of cytokine secretion in vivo, because high levels of cytokine secretion by CAR-T cells in patients often lead to cytokine release syndrome. The decreased cytokine production of the CAR-T cells of the present invention may allow multiple applications.

Combination therapy with several CAR mutants, inhibitors of immune checkpoints, tumor microenvironment can be used to increase activity of single CAR.

The following examples further illustrate the present invention. These examples are intended merely to be illustrative of the present invention and are not to be construed as being limiting.

### EXAMPLES

### Materials and Methods

### Example 1. Cell Lines

A1847, SKOV-3, C30 ovarian cells and BxPC3 pancreatic cancer cells were cultured in DMEM (GE Healthcare, Chicago, Ill.) containing 10% FBS (AmCell, Mountain View, Calif.). Human peripheral blood mononuclear cells (PBMC) were isolated by density sedimentation over Ficoll-Paque (GE Healthcare). HEK293FT cells were a gift from AlStem (Richmond, Calif.) and were cultured in DMEM containing 10% FBS. All cell lines were authenticated by flow cytometry in our laboratory, using cell-specific surface markers.

### Example 2. Mesothelin CAR Constructs

The Mesothelin P4 human antibody (Lanitis, et al. (2012), Mol Ther 20, 633-643) was inserted into a second-generation CAR cassette containing a signaling peptide from human CD8, a hinge region from CD8 alpha, transmembrane domain and costimulatory domains from CD28, and the CD3 zeta activation domain; this CAR is herein called the Meso TM28-28 WT-Z (Z denotes CD3 zeta) CAR. The mutant Meso CAR was also generated with duplicated motif YMNM (191-194 amino acids of CD28 protein) and also duplicated motif PYAP (208-211 amino-acids of CD28 protein), called Mesothelin-DD28-Z (DD, duplicated domain of CD28). In addition, T195P mutation was introduced into the above CD28 mutant domains generating triple mutant of CD28 domain (called Mesothelin-DDT195P28-Z). In some CAR constructs Flag tag was added after P4 ScFv, and two serines changed to cysteines in the CD8 hinge region.

### Example 3. EGFR CAR Constructs

The EGFR C10 human low affinity antibody sequence (Liu et al (2015) Cancer Res 75, 3596-3607) was used to generate CAR. The EGFR scFV was subcloned using Nhe I and Xho I sites into Mesothelin constructs. The structures were the same as Mesothelin CAR containing a signaling peptide from human CD8, a hinge region from CD8 alpha, transmembrane domain and costimulatory domains from CD28 either wild type or with DD28 or DDT195P mutation (described above), and the CD3 zeta activation domain.

### Example 4. Generation of CAR-Encoding Lentivirus

DNAs encoding the CARs were synthesized and subcloned into a third-generation lentiviral vector, Lenti CMV-MCS-EF1a-puro by Syno Biological (Beijing, China). All CAR lentiviral constructs were sequenced in both directions to confirm CAR sequence and used for lentivirus production. Ten million growth-arrested HEK293FT cells (Thermo Fisher) were seeded into T75 flasks and cultured overnight, then transfected with the pPACKH1 Lentivector Packaging mix (System Biosciences, Palo Alto, Calif.) and 10 μg of each lentiviral vector using the CalPhos Transfection Kit (Takara, Mountain View, Calif.). The next day the medium was replaced with fresh medium, and 48 h later the lentivirus-containing medium was collected. The medium was cleared of cell debris by centrifugation at 2100 g for 30 min. The virus particles were collected by centrifugation at 112,000 g for 100 min, suspended in AIM V medium, aliquoted and frozen at −80° C. The titers of the virus preparations were determined by quantitative RT-PCR using the Lenti-X qRT-PCR kit (Takara) according to the manufacturer's protocol and the 7900HT thermal cycler (Thermo Fisher). The lentiviral titers were >1×10⁸ pfu/ml.

### Example 5. Generation and Expansion of CAR-T Cells

PBMC were suspended at 1×10⁶ cells/ml in AIM V-AlbuMAX medium (Thermo Fisher) containing 10% FBS and 300 U/ml IL-2 (Thermo Fisher), mixed with an equal number (1:1 ratio) of CD3/CD28 Dynabeads (Thermo Fisher), and cultured in non-treated 24-well plates (0.5 ml per well). At 24 and 48 hours, lentivirus was added to the cultures at a multiplicity of infection (MOI) of 5, along with 1 μl of TransPlus transduction enhancer (AlStem). As the T cells proliferated over the next two weeks, the cells were counted every 2-3 days and fresh medium with 300 U/ml IL-2 was added to the cultures to maintain the cell density at 1-3×10⁶ cells/ml.

### Example 6. Flow Cytometry

To measure CAR expression, 0.5 million cells were suspended in 100 μl of buffer (PBS containing 0.5% BSA) and incubated on ice with 1 μl of human serum (Jackson Immunoresearch, West Grove, Pa.) for 10 min. Then 1 μl of allophycocyanin (APC)-labeled anti-CD3 (eBioscience, San Diego, Calif.), 2 μl of 7-aminoactinomycin D (7-AAD, BioLegend, San Diego, Calif.), and 2 μl of either phycoerythrin (PE)-labeled anti-FLAG or its isotype control PE rat IgG2a (both from BioLegend) was added, and the cells were incubated on ice for 30 min. The cells were rinsed with 3 ml of buffer, then suspended in buffer and acquired on a FACSCalibur (BD Biosciences). Cells were analyzed first for light scatter versus 7-AAD staining, then the 7-AAD⁻ live gated cells were plotted for CD3 staining versus FLAG staining or isotype control staining. For no Flag-tagged Mesothelin CAR constructs, biotin-labeled polyclonal goat anti-human-F(ab)2 antibodies (Life Technologies) that detect Meso scFv and biotin-labeled normal polyclonal goat IgG antibodies (Life Technologies) were used for FACS staining to detect CAR expression.

### Example 7. Real-Time Cytotoxicity Assay (RTCA)

Adherent target cells (A1847 or C30) were seeded into 96-well E-plates (Acea Biosciences, San Diego, Calif.) at 1×10⁴ cells per well and monitored in culture overnight with the impedance-based real-time cell analysis (RTCA) iCELLigence system (Acea Biosciences). The next day, the medium was removed and replaced with AIM V-AlbuMAX medium containing 10% FBS±1×10⁵ effector cells (CAR-T cells or non-transduced T cells), in triplicate. The cells in the E-plates were monitored for another 2-3 days with the RTCA system, and impedance was plotted over time. Cytolysis was calculated as (impedance of target cells without effector cells—impedance of target cells with effector cells)×100/impedance of target cells without effector cells.

### Example 8. Cytokine Secretion Assay

The target cells were cultured with the effector cells (CAR-T cells or non-transduced T cells) at a 1:1 ratio (1×10⁴ cells each) in U-bottom 96-well plates with 200 μl of AIM V-AlbuMAX medium containing 10% FBS, in triplicate. After 16 h the top 150 μl of medium was transferred to V-bottom 96-well plates and centrifuged at 300 g for 5 min to pellet any residual cells. The top 120 μl of supernatant was transferred to a new 96-well plate and analyzed by ELISA for human IFN-gamma and IL-2 levels using kits from Thermo Fisher according to the manufacturer's protocol.

### Results

### Example 9. CAR Constructs

A human Mesothelin-specific CAR was constructed consisting of the P4 human Mesothelin single-chain variable fragment (scFv); hinge, transmembrane and co-stimulation domains from human CD28; and the activation domain of human CD3 zeta (FIG. 2). A “mock” CAR with an scFv specific for an intracellular protein—and thus not reactive with intact cells—was constructed in the same manner (FIG. 2). In addition, the 8-amino acid FLAG epitope was inserted between the scFv and hinge region of the Mesothelin-specific CAR. Sequences for each CAR were transferred into a lentiviral vector downstream of the cytomegalovirus immediate-early promoter, and CAR-encoding lentivirus particles were produced by transient transfection of HEK293FT cells. The viruses were added at an MOI of 5 to activated human T cells, which were then cultured with IL-2 for 14 days. The CAR-T cells effectively expanded during this time and expressed CAR more than 40% that was analyzed by flow cytometry (not shown).

To test the effect of CD mutation with other scFv, we generated EGFR-CAR-T cells with wild type and mutant CD28 domain with DD28 and DD28T195P mutations by sub-cloning EGFR scFv into Nhe I and Xho I sites instead of Mesothelin scFv of Meso-CAR constructs.

### Example 10. Sequences of Mesothelin CAR Construct

The amino acid sequences of each segment of Meso-(±Flag)-TM28-28 DD-Z, Meso-(±Flag)-TM28-28 DD T195P-z are shown as follows. The sequence of each segment can be replaced by amino acid sequence with at least 95% identity.

<Signal peptide human CD8>
SEQ ID NO: 11
M A L P V T A L L L P L A L L L H A A R P (SEQ ID
No. 11)
<NheI site> AS
ScFv (VH-Linker-VL) of anti-mesothelin P4
<VH>
SEQ ID NO: 12
Q V Q L Q Q S G P G L V T P S Q T L S L T C A I S
G D S V S S N S A T W N W I R Q S P S R G L E W L
G R T Y Y R S K W Y N D Y A V S V K S R M S I N P
D T S K N Q F S L Q L N S V T P E D T A V Y Y C A
R G M M T Y Y Y G Met D V W G Q G T T V T V S S
<linker>
SEQ ID NO: 13
G I L G S G G G G S G G G G S G G G G S,
or
SEQ ID NO: 14
G G G G S G G G G S G G G G S,
<VL>
SEQ ID NO: 15
QPVLTQSSSLSASPGASASLTCTLRSGINVGPYRIYWYQQKPGSPPQYLL
NYKSDSDKQQGSGVPSRFSGSKDASANAGVLLISGLRSEDEADYYCMIWH
SSAAVFGGGTQLTVLS
<Xho I site> LE
<Flag tag> is optionally inserted after VL,
SEQ ID NO: 16
DYKDDDDK
<CD8 hinge SS>
SEQ ID NO: 17
K P T T T P A P R P P T P A P T I A S Q P L S L R
P E A S R P A A G G A V H T R G L D F A S D K P
<CD8 hinge CC> with two S (serines) changed to
cysteine (C), underlined and bolded, used in
Flag tagged constructs only,
SEQ ID NO: 18
K P T T T P A P R P P T P A P T I A S Q P L S L R
P E A C R P A A G G A V H T R G L D F A C D K P
<CD28 Protein>
SEQ ID NO: 19
The entire CD28 protein sequence is shown here.
The transmembrane segment TM28 is italicized, the
two binding motifs YMNM and PYAP are bolded, T195
is underlined.
MLRLLLALNL FPSIQVTGNK ILVKQSPMLV AYDNAVNLSC
KYSYNLFSRE FRASLHKGLD SAVEVCVVYG NYSQQLQVYS
KTGFNCDGKL GNESVTFYLQ NLYVNQTDIY FCKIEVMYPP
PYLDNEKSNG TIIHVKGKHL CPSPLFPGPS KPFWVLVVVG
GVLACYSLLV TVAFIIFWVR SKRSRLLHSD YMNMTPRRPG
PTRKHYQPYAPPRDFAAYRS
<Transmembrane Domain TM28>
SEQ ID NO: 20
F W V L V V V G G V L A C Y S L L V T V A F I I F
W V
<Co-stimulating domain CD28-WT>
SEQ ID NO: 21
R S K R S R L L H S D Y M N M T P R R P G P T R K
H Y Q P Y A P P R D F A A Y R S
<Co-stimulating domain CD28 DD>
SEQ ID NO: 22
In one embodiment of the present invention, the
two binding motifs (YMNM and PYAP) are immediately
repeated (bolded) as shown in the following
sequence:
F W V L V V V G G V L A C Y S L L V T V A F I I F
W V R S K R S R L L H S D Y M N M Y M N M T P R R
P G P T R K H Y Q P Y A P P Y A P P R D F A A Y R
S
<Co-stimulating domain CD28 DD T195P>
SEQ ID NO: 23
F W V L V V V G G V L A C Y S L L V T V A F I I F
W V R S K R S R L L H S D Y M N M Y M N M P P R R
P G P T R K H Y Q P Y A P P Y A P P R D F A A Y R
S
<Activation domain is CD3-zeta>. In case of Meso
or EGFR constructs with mutation in CD28 domain,
CD3-zeta activation domain had deletion of Q (bold
underlined).
SEQ ID NO: 24
R V K F S R S A D A P A Y Q Q G Q N Q L Y N E L N
L G R R E E Y D V L D K R R G R D P E Met G G K P
Q R R K N P Q E G L Y N E L Q K D K M A E A Y S E
I G M K G E R R R G K G H D G L Y Q G L S T A T K
D T Y D A L H M Q A L P P R

### Example 11. Sequences of EGFR CAR Construct

The amino acid sequences of each segment of EGFR-TM28-28 DD-Z and EGFR TM28-28 DD T195P-Z are shown as follows.

EGFR scFv is composed with EGFR VH-Linker-EGFR VL.

<EGFR VH>
SEQ ID NO: 25
E V Q L V Q S G A E V K K P G S S V K V S C K A S
G G T F S S Y A I S W V R Q A P G Q G L E W M G G
I I P I F G T A N Y A Q K F Q G R V T I T A D E S
T S T A Y M E L S S L R S E D T A V Y Y C A R E E
G P Y C S S T S C Y G A F D I W G Q G T L V T V S
S
<Linker>
SEQ ID NO: 26
GGGGS GGGGS GGGGS
<EGFR VL>
SEQ ID NO: 27
Q S V L T Q D P A V S V A L G Q T V K I T C Q G D
S L R S Y F A S W Y Q Q K P G Q A P T L V M Y A R
N D R P A G V P D R F S G S K S G T S A S L A I S
G L Q S E D E A D Y Y C A A W D D S L N G Y L F G
A G T K L T V L

The amino acid sequences of EGFR construct were similar to mesothelin construct as shown in Example 10, except EGFR scFv (instead of mesothelin scFv) was inserted into Nhe I and Xho I sites of either CD28WT, or CD28DD, or CD28DDT195P mutant CAR constructs. No Flag tag was used in the EGFR constructs.

### Example 12. Cytotoxicity of Mesothelin-CAR-T Cells on Human Ovarian and Pancreatic Cancer Cell Lines

The ability of Mesothein-CAR-T cells to kill mesothelin-bearing target cells was tested using human ovarian cancer cell lines: A1847, which endogenously expresses mesothelin, and mesothelin-positive pancreatic BxPC3 cancer cell line. Cytolysis was detected using the real-time cell analysis (RTCA) iCELLigence system, which measures the impedance of the target cell monolayer over time; as the target cells are killed by the effector cells, the impedance decreases. The Mesothelin-CAR-T cells exhibited significant cytolytic activity against A1847 ovarian cancer cells (FIG. 3).

In addition, RTCA with mesothelin-positive cells demonstrated increased cytolytic activity of Meso-Flag-TM28-28 DDT195P-Z and Meso-Flag-TM28-28 DD-Z CAR-T cells compared with wild type Meso-TM28-28WT-Z CAR-T cells. The Real-time highly sensitive cytotoxicity assay demonstrated significantly higher activity of Meso-Flag-TM28-28 DD T195P-Z and Meso Flag-TM28-28 DD-Z CAR-T cells versus Meso-TM28-28 WT-Z CAR-T cells and Meso Flag-4-1BB-Z CAR-T cells with ovarian target cell line A1847 (FIG. 3). The difference was significant, p<0.05 between cells. 10:1 Effector to Target Ratio was used. Meso Flag-TM8-4-11 BB-Z CAR-T cells, where CD8 trans-membrane domain was used, were less cytolytic compared to same with same construct with CD28 transmembrane domain (FIG. 3). Meso Flag TM28-28WT-z CAR-T cells were more cytolytic than Meso-Flag-4-TM28-1BB-Z cells with CD28 TM domain. The mutant with duplicated domains and T195P mutation had the highest cytolytic activity against A1847 cells (FIG. 3). The similar result was observed with Mesothelin-CAR constructs with no Flag tag (not shown).

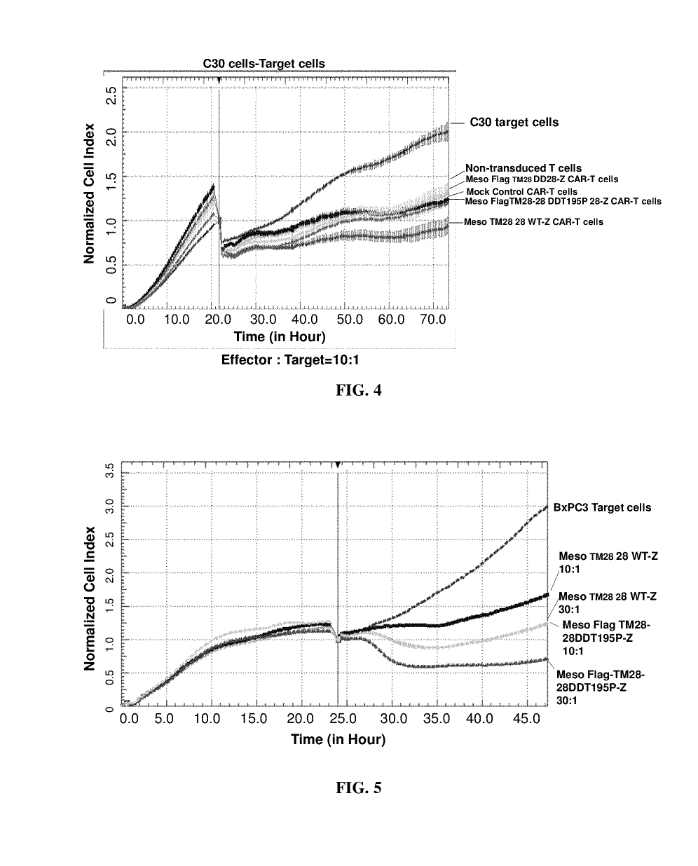

The cytolytic activity of mesothelin CAR-T cells was not observed against Mesothelin-negative cancer cells (FIG. 4), in which 10:1 dilution of effector (CAR-T cells) to target cells (C30 cells) was used. Thus, both mesothelin-CAR-T cells with different CD28 mutations (DD or DDT195P) exhibited strong mesothelin-dependent cytolytic activity.

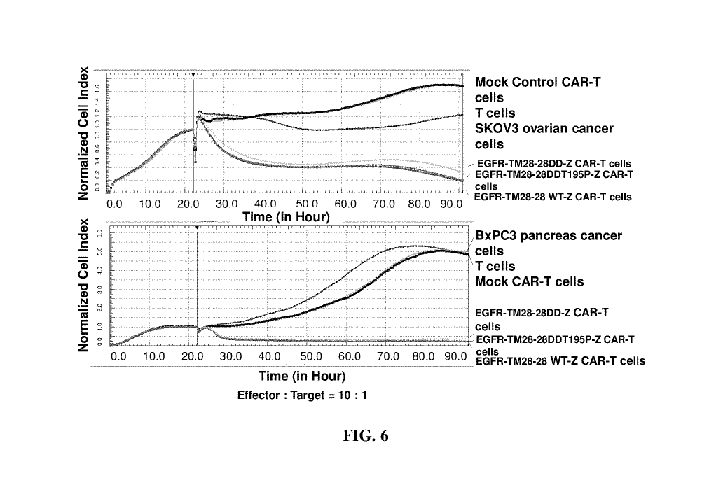

FIG. 5 shows that mesothelin CAR-T cells with mutant CD28 domain are highly cytolytic against mesothelin-positive pancreatic cancer cells. RTCA assay with Meso-Flag-TM28-28 DD T195P-Z domain mutation CAR-T cells and Meso TM28-28 WT-Z CAR-T cells against BxPC3 pancreatic cancer cells is shown. The same high cytolytic activity of mesothelin-Flag mutant with duplicated domains of CD28 and T195P mutation in CD28 domain was observed in BxPC3 pancreatic cancer cells (FIG. 5) demonstrating the same effect of Mesothelin CAR-T cells in different type of cancer cells (FIG. 5). The effector CAR-T cells were used at 10:1 and 30:1 dilution ratio to target BxPC3 cells demonstrating dose-dependent increase of cytolytic activity of CAR-T cells against target cells.

### Example 13. Cytotoxicity of EGFR-CAR-T Cells on Human Ovarian and Pancreatic Cancer Cell Lines

The RTCA assay with EGFR-CAR-T cells as effector cells and EGFR-positive SKOV-3 ovarian or BxPC-3 pancreatic cancer target cells demonstrates that EGFR-TM28-28 DD-Z, TM28-28 DD T195P-Z, and EGFR-TM28-28 WT CAR-T cells all expressed high cytotoxic activity in SKOV-3 cells and BxPC3 cells (FIG. 6). The same effect was observed with A431 cell line, and no increased RTCA activity was observed with EGFR-negative MCF-7 target cells (not shown). Thus, CD28 mutation either increases activity of CAR-T cells or maintains the same activity as CAR-T cells with wild type CD28.

### Example 14. Cytokine Secretion of Mesothelin CAR-T Cells

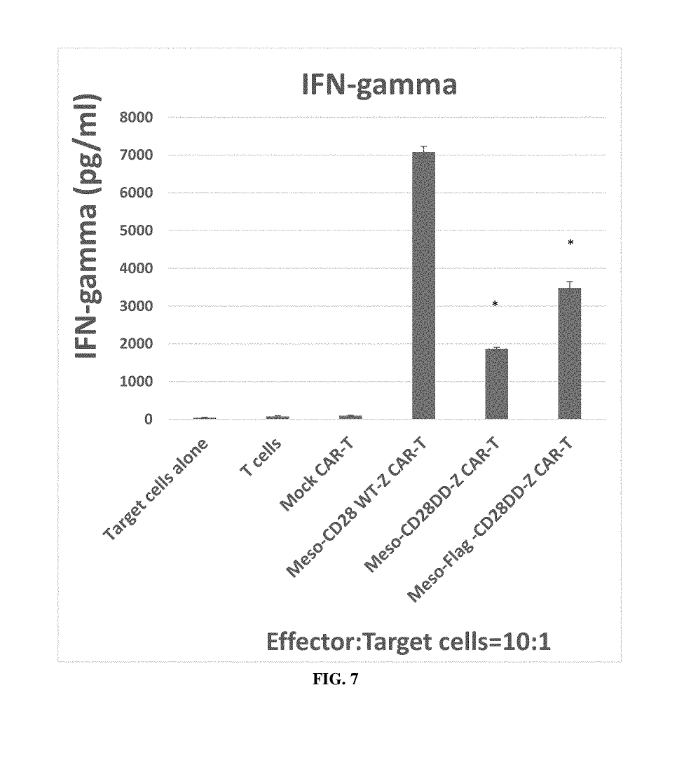

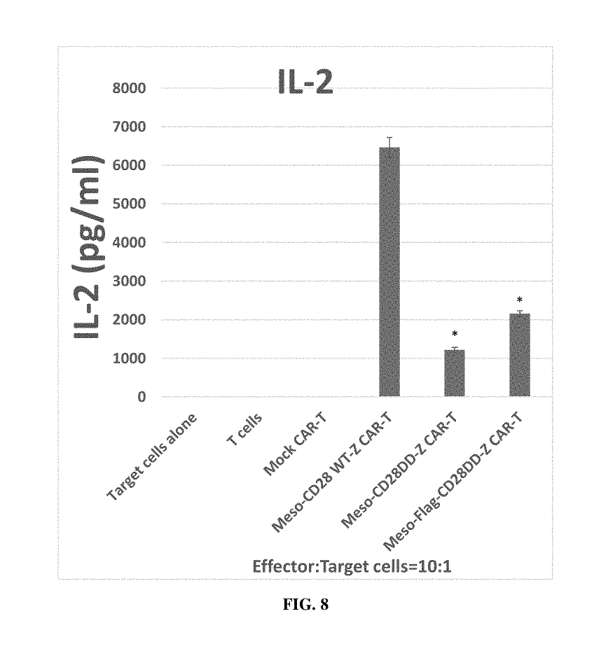

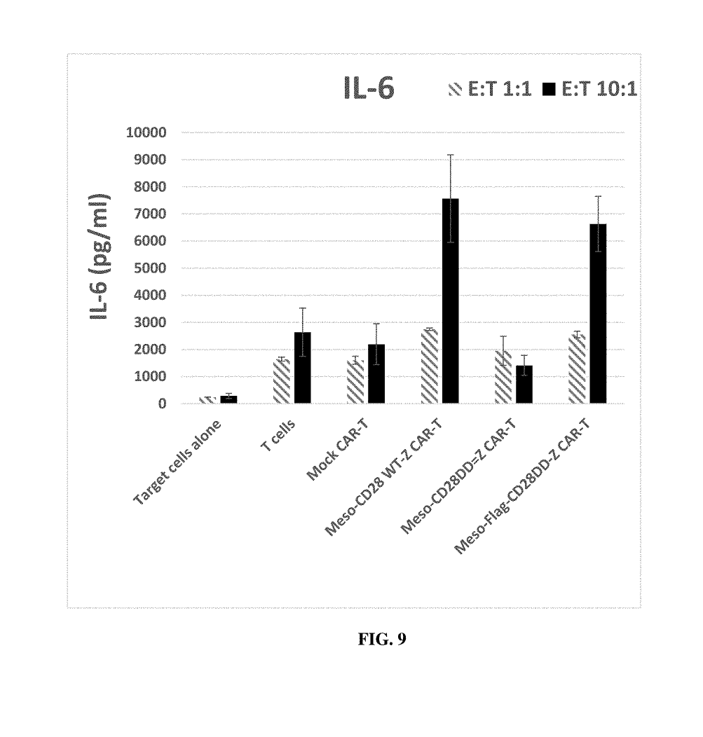

The CAR-T cells were evaluated for their ability to produce IFN-γ and IL-2 in response to mesothelin-positive target cells. Meso-Flag-TM28-28 DD-Z or Meso-TM28-28 DD-Z CAR-T cells produced significantly lower levels of IFN-γ and IL-2 (FIGS. 7 and 8) when cultured with mesothelin-positive A1847 target cells, in compared with wild type Meso-TM28-WT-Z. As expected, the mock CAR-T cells and non-transduced T cells did not produce significant levels of either cytokine when cultured with target cell lines. Meso-Flag-TM28-28 DD-Z or Meso-TM28-28 DD-Z CAR-T cells also produced lower levels of 11-6, in compared with wild type Meso-TM28-WT-Z, when cultured with A1847 cells (FIG. 9). Thus, Meso-TM28-28 DD-Z and Meso-Flag-TM28-28 DD-Z CAR-T cells secreted significantly less IFN-γ, IL-2 and IL-6 in response to mesothelin-positive cells suggesting safer CAR-T cells in clinic.

### Example 15. Cytokine Secretion of EGFR CAR-T Cells

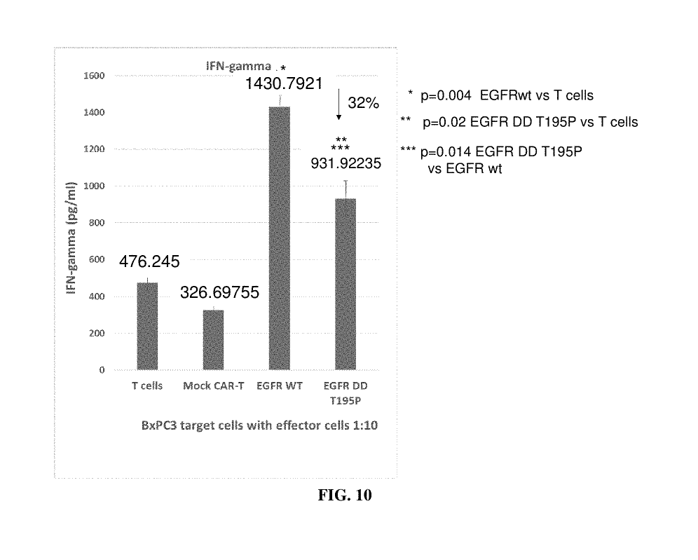

FIG. 10 shows a significantly decreased secretion of IFN-gamma by EGFR-TM28-28 DDT195P-Z cells against BxPC3 cells compared to EGFR-TM28-28WT-Z CAR-T cells, with p<0.05.

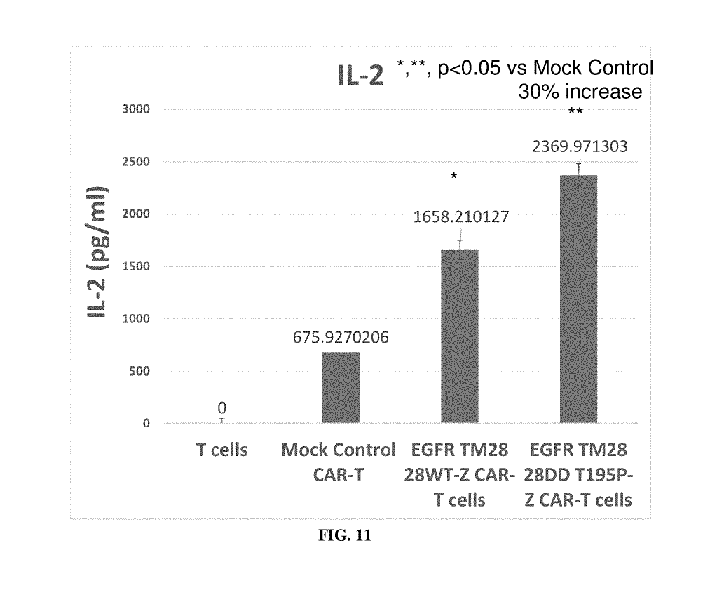

FIG. 11 shows a significantly increased secretion of IL-2 by EGFR-TM28-28 DDT195P-Z cells against BxPC3 cells compared to EGFR-TM28-28WT-Z CAR-T cells, with p<0.05.

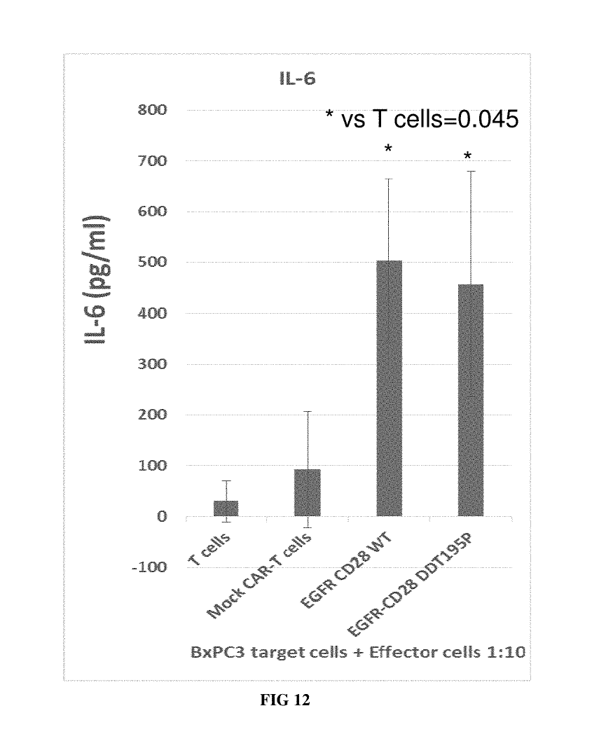

FIG. 12 shows similar secretion of IL-6 by EGFR-TM28-28 DDT195P-Z cells against BxPC3 cells compared to EGFR-TM28-28WT-Z CAR-T cells, with p<0.05.

It is to be understood that the foregoing describes preferred embodiments of the present invention and that modifications may be made therein without departing from the scope of the present invention as set forth in the claims.

What is claimed is:

1. A chimeric antigen receptor fusion protein comprising from N-terminus to C-terminus:
(i) a single-chain variable fragment (scFv) having activity against a tumor antigen,
(ii) a transmembrane domain,
(iii) at least one co-stimulatory domain having one or more binding motif immediately repeated at least one time, and
(iv) an activating domain.

2. The fusion protein according to claim 1, wherein the tumor antigen is selected from the group consisting of: mesothelin, BCMA, VEGFR-2, CD4, CD5, CD19, CD20, CD30, CD22, CD24, CD25, CD28, CD30, CD33, CD38, CD47, CD52, CD56, CD80, CD81, CD86, CD123, CD138, CD171, CD276, B7H4, CD133, EGFR, GPC3; PMSA, CD3, CEACAM6, c-Met, EGFRvIII, ErbB2/HER-2, ErbB3/HER3, ErbB4/HER-4, EphA2, Eph10A, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, NGFR, MCAM, TNFR1, TNFR2, PD1, PD-L1, PD-L2, HVEM, MAGE-A, NY-ESO-1, PSMA, RANK, ROR1, ROR-2, TNFRSF4, CD40, CD137, TWEAK-R, LTPR, LIFRP, LRP5, MUC1, TCRa, TCRp, TLR7, TLR9, PTCH1, WT-1, Robol, a, Frizzled, OX40, CD79b, and Notch-1-4.

3. The fusion protein according to claim 2, wherein the tumor antigen is mesothelin or EGFR.

4. The fusion protein according to claim 1, wherein the co-stimulatory domain is selected from the group consisting of CD28, 4-1BB, ICOS-1, CD27, OX-40, GITR, and DAP10.

5. The fusion protein according to claim 4, wherein the co-stimulatory domain is from CD28.

6. The fusion protein according to claim 5, comprising the binding motif YMNM repeated 1, 2, 3, or 4 times.

7. The fusion protein according to claim 5, comprising the binding motif PYAP repeated 1, 2, 3, or 4 times.

8. The fusion protein according to claim 5, comprising a mutation from T to P at amino acid position 195 of the CD28 protein.

9. The fusion protein according to claim 5, comprising a binding motif PYAP immediately repeated once and a binding motif PYAP immediately repeated once, and a mutation from T to P at amino acid position 195 of the CD28 protein.

10. The fusion protein according to claim 1, wherein scFv comprises V_{H} and V_{L}, and the fusion protein further comprises a FLAG tag N-terminus to V_{H}, or between V_{H} and V_{L}, or between V_{L} and the transmembrane domain.

11. The fusion protein according to claim 1, which has the amino acid sequence of SEQ ID NO: 28, referred as Meso-Flag-TM28-28-DD-Z in FIG. 2, or having at least 95% identity in each segment.

12. The fusion protein according to claim 1, which has the amino acid sequence of SEQ ID NO: 29, referred as Meso-Flag-TM28-28 DD T195P-Z in FIG. 2, or having at least 95% identity in each segment.

13. The fusion protein according to claim 1, which has the amino acid sequence of SEQ ID NO: 30, referred as EGFR-TM28-28 DD-Z in FIG. 2, or having at least 95% identity in each segment.

14. The fusion protein according to claim 1, which has the amino acid sequence of SEQ ID NO: 31, referred as EGFR-TM28-28 DD T195P-Z in FIG. 2, or having at least 95% identity in each segment.

15. The fusion protein according to claim 1, which has the amino acid sequence of SEQ ID NO: 32 or 33.

16. A nucleic acid sequence encoding the fusion protein of claim 1.

17. T cells modified to express the chimeric antigen receptor fusion protein of claim 1.


SEQUENCE LISTING (33 sequences)

SEQ ID NO: 1 - DNA, 8 bp, Artificial Sequence
  synthetic
dykddddk

SEQ ID NO: 2 - protein, 4 aa, Homo sapiens
YMNM

SEQ ID NO: 3 - protein, 4 aa, Homo sapiens
PYAP

SEQ ID NO: 4 - protein, 4 aa, Homo sapiens
QEED

SEQ ID NO: 5 - protein, 4 aa, Homo sapiens
EEEE

SEQ ID NO: 6 - protein, 4 aa, Homo sapiens
YMFM

SEQ ID NO: 7 - protein, 9 aa, Homo sapiens
TPIQEEQAD

SEQ ID NO: 8 - protein, 4 aa, Homo sapiens
YINM

SEQ ID NO: 9 - protein, 5 aa, Homo sapiens
PIQED

SEQ ID NO: 10 - protein, 11 aa, Homo sapiens
SCQFPEEERGE

SEQ ID NO: 11 - protein, 21 aa, Homo sapiens
MALPVTALLLPLALLLHAARP

SEQ ID NO: 12 - protein, 127 aa, Homo sapiens
QVQLQQSGPGLVTPSQTLSLTCAISGDSVSSNSATWNWIRQSPSRGLEWLGRTYYRSKWYNDYAVSVKSRMETSINPDTSKNQFSLQLNSVTPEDTAVYYCARGMMTYYYGMETDVWGQGTTVTVSS

SEQ ID NO: 13 - protein, 20 aa, Artificial Sequence
  synthetic
GILGSGGGGSGGGGSGGGGS

SEQ ID NO: 14 - protein, 15 aa, Artificial Sequence
  synthetic
GGGGSGGGGSGGGGS

SEQ ID NO: 15 - protein, 116 aa, Homo sapiens
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 16 - protein, 8 aa, Artificial Sequence
  synthetic
DYKDDDDK

SEQ ID NO: 17 - protein, 49 aa, Homo sapiens
KPTTTPAPRPPTPAPTIASQPLSLRPEASRPAAGGAVHTRGLDFASDKP

SEQ ID NO: 18 - protein, 49 aa, Homo sapiens
KPTTTPAPRPPTPAPTIASQPLSLRPEACRPAAGGAVHTRGLDFACDKP

SEQ ID NO: 19 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 20 - protein, 27 aa, Homo sapiens
FWVLVVVGGVLACYSLLVTVAFIIFWV

SEQ ID NO: 21 - protein, 41 aa, Homo sapiens
RSKRSRLLHSDYMNMTPRRPGPTRKHYQPYAPPRDFAAYRS

SEQ ID NO: 22 - protein, 76 aa, Homo sapiens
FWVLVVVGGVLACYSLLVTVAFIIFWVRSKRSRLLHSDYMNMYMNMTPRRPGPTRKHYQPYAPPYAPPRDFAAYRS

SEQ ID NO: 23 - protein, 76 aa, Homo sapiens
FWVLVVVGGVLACYSLLVTVAFIIFWVRSKRSRLLHSDYMNMYMNMPPRRPGPTRKHYQPYAPPYAPPRDFAAYRS

SEQ ID NO: 24 - protein, 115 aa, Homo sapiens
RVKFSRSADAPAYQQGQNQLYNELNLGRREEYDVLDKRRGRDPEMETGGKPQRRKNPQEGLYNELQKDKMAEAYSEIGMKGERRRGKGHDGLYQGLSTATKDTYDALHMQALPPR

SEQ ID NO: 25 - protein, 130 aa, Homo sapiens
EVQLVQSGAEVKKPGSSVKVSCKASGGTFSSYAISWVRQAPGQGLEWMETGGIIPIFGTANYAQKFQGRVTITADESTSTAYMETELSSLRSEDTAVYYCAREEGPYCSSTSCYGAFDIWGQGTLVTVSS

SEQ ID NO: 26 - protein, 15 aa, Artificial Sequence
  synthetic
GGGGSGGGGSGGGGS

SEQ ID NO: 27 - protein, 110 aa, Homo sapiens
QSVLTQDPAVSVALGQTVKITCQGDSLRSYFASWYQQKPGQAPTLVMETYARNDRPAGVPDRFSGSKSGTSASLAISGLQSEDEADYYCAAWDDSLNGYLFGAGTKLTVL

SEQ ID NO: 28 - protein, 529 aa, Homo sapiens
MALPVTALLLPLALLLHAARPASQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSLEDYKDDDDKKPTTTPAPRPPTPAPTIASQPLSLRPEACRPAAGGAVHTRGLDFACDKPFWVLVVVGGVLACYSLLVTVAFIIFWVRSKRSRLLHSDYMNMYMNMTPRRPGPTRKHYQPYAPPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 529 aa, Homo sapiens
MALPVTALLLPLALLLHAARPASQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSLEDYKDDDDKKPTTTPAPRPPTPAPTIASQPLSLRPEACRPAAGGAVHTRGLDFACDKPFWVLVVVGGVLACYSLLVTVAFIIFWVRSKRSRLLHSDYMNMYMNMPPRRPGPTRKHYQPYAPPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 517 aa, Homo sapiens
MALPVTALLLPLALLLHAARPASEVQLVQSGAEVKKPGSSVKVSCKASGGTFSSYAISWVRQAPGQGLEWMETGGIIPIFGTANYAQKFQGRVTITADESTSTAYMETELSSLRSEDTAVYYCAREEGPYCSSTSCYGAFDIWGQGTLVTVSSGGGGSGGGGSGGGGSQSVLTQDPAVSVALGQTVKITCQGDSLRSYFASWYQQKPGQAPTLVMETYARNDRPAGVPDRFSGSKSGTSASLAISGLQSEDEADYYCAAWDDSLNGYLFGAGTKLTVLLEKPTTTPAPRPPTPAPTIASQPLSLRPEACRPAAGGAVHTRGLDFACDKPFWVLVVVGGVLACYSLLVTVAFIIFWVRSKRSRLLHSDYMNMYMNMTPRRPGPTRKHYQPYAPPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 517 aa, Homo sapiens
MALPVTALLLPLALLLHAARPASEVQLVQSGAEVKKPGSSVKVSCKASGGTFSSYAISWVRQAPGQGLEWMETGGIIPIFGTANYAQKFQGRVTITADESTSTAYMETELSSLRSEDTAVYYCAREEGPYCSSTSCYGAFDIWGQGTLVTVSSGGGGSGGGGSGGGGSQSVLTQDPAVSVALGQTVKITCQGDSLRSYFASWYQQKPGQAPTLVMETYARNDRPAGVPDRFSGSKSGTSASLAISGLQSEDEADYYCAAWDDSLNGYLFGAGTKLTVLLEKPTTTPAPRPPTPAPTIASQPLSLRPEASRPAAGGAVHTRGLDFASDKPFWVLVVVGGVLACYSLLVTVAFIIFWVRSKRSRLLHSDYMNMYMNMPPRRPGPTRKHYQPYAPPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 521 aa, Homo sapiens
MALPVTALLLPLALLLHAARPASQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSLEKPTTTPAPRPPTPAPTIASQPLSLRPEASRPAAGGAVHTRGLDFASDKPFWVLVVVGGVLACYSLLVTVAFIIFWVRSKRSRLLHSDYMNMYMNMTPRRPGPTRKHYQPYAPPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 521 aa, Homo sapiens
MALPVTALLLPLALLLHAARPASQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSLEKPTTTPAPRPPTPAPTIASQPLSLRPEASRPAAGGAVHTRGLDFASDKPFWVLVVVGGVLACYSLLVTVAFIIFWVRSKRSRLLHSDYMNMYMNMPPRRPGPTRKHYQPYAPPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR